(19) United States
(12) Patent Application Publication
(10) Pub. No.: US 2015/0182198 A1
(43) Pub. Date: Jul. 2, 2015

(54) SYSTEM AND METHOD FOR DISPLAYING ULTRASOUND IMAGES

(71) Applicant: General Electric Company, Schenectady, NY (US)

(72) Inventor: Thomas Sabourin, Milwaukee, WI (US)

(73) Assignee: General Electric Company, Schenectady, NY (US)

(21) Appl. No.: 14/141,881

(22) Filed: Dec. 27, 2013

(51) Int. Cl. A61B 8/00; A61B 8/08; A61B 19/00; A61B 8/14

(52) U.S. Cl. CPC A61B 8/463; A61B 8/14; A61B 8/469; A61B 8/4254; A61B 8/4416; A61B 8/5207; A61B 19/5244; A61B 19/5212; A61B 2019/5248; A61B 2019/5251; A61B 2019/5265; A61B 2019/5255

(57) ABSTRACT

An ultrasound imaging system comprises a probe configured to transmit ultrasound signals toward a target region of interest (ROI) and receive returned echo data from the target ROI. The system further comprises a sensor for generating a signal relating to a probe orientation, a display for displaying an image of the target ROI and a processor connected the probe for receiving the echo data and generating the image of the target ROI. The processor is further configured to receive the probe orientation signal and display the image of the target ROI based on the probe orientation signal.

### BACKGROUND OF THE INVENTION

The subject matter disclosed herein relates generally to an ultrasound imaging system and a method for orientating the ultrasound image displayed.

In the field of medical ultrasound imaging, a probe, comprising a transducer array, is typically used to transmit ultrasound energy into a target, such as a patient, and to detect reflected ultrasound energy from the target. Based on the energy and timing of the reflected ultrasound waves, it is possible to determine detailed information about a region of interest (ROI) inside the target. The information may be used to generate images and/or quantitative data such as blood flow direction or rate of flow.

Generally, the processed ultrasound images are displayed at 0 degrees, meaning that the axis bisecting the displayed ROI is a vertical gravitational axis or a y-axis. For an inexperienced user, it may be difficult to comprehend the spatial relationship between a target ROI being scanned and the orientation of the displayed ROI image. Additionally, since the field of view provided by the transducer geometry only provides a subset of the slice the anatomy of interest, it can be a challenge for an inexperienced user to find and visualize what they are looking for. To further complicate challenges faced by an inexperienced user, the display orientation of a portable or handheld ultrasound system may be variable and inconsistent. As a result of these challenges, increases in scan times and overall exam length may produce workflow inefficiencies.

Therefore, a system and method for displaying ultrasound images having the orientation of the displayed anatomy change based on the orientation of the probe and/or the device is desired.

The above-mentioned shortcomings, disadvantages and problems are addressed herein which will be understood by reading and understanding the following specification.

### BRIEF DESCRIPTION OF THE INVENTION

In an embodiment, an ultrasound imaging system comprises a probe configured to transmit ultrasound signals toward a target region of interest (ROI) and receive returned echo data from the target ROI. The system further comprises a sensor for generating a signal relating to a probe orientation, a display for displaying an image of the target ROI and a processor connected to the probe for receiving the echo data and generating the image of the target ROI. The processor is further configured to receive the probe orientation signal and display the image of the target ROI based on the probe orientation signal.

In another embodiment, a method of displaying an ultrasound image comprises scanning with a probe a target region of interest (ROI) and receiving echo data from the ROI, and sensing with a sensor a probe orientation. The method further comprises generating with a processor an image of the ROI, and displaying with a display the image of the ROI based on the probe orientation.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram on an ultrasound imaging system in accordance with an embodiment;

FIG. 2 is schematic representation of the ultrasound probe in accordance with the embodiment of FIG. 1, scanning a target;

FIG. 3 is a schematic representation of a displayed ROI image in accordance with an embodiment;

FIG. 4 is a schematic representation of a displayed ROI image in accordance with an embodiment; and

FIG. 5 is a flowchart of a method in accordance with the embodiment of FIG. 3; and

FIG. 6 is a flowchart of a method in accordance with the embodiment of FIG. 4.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

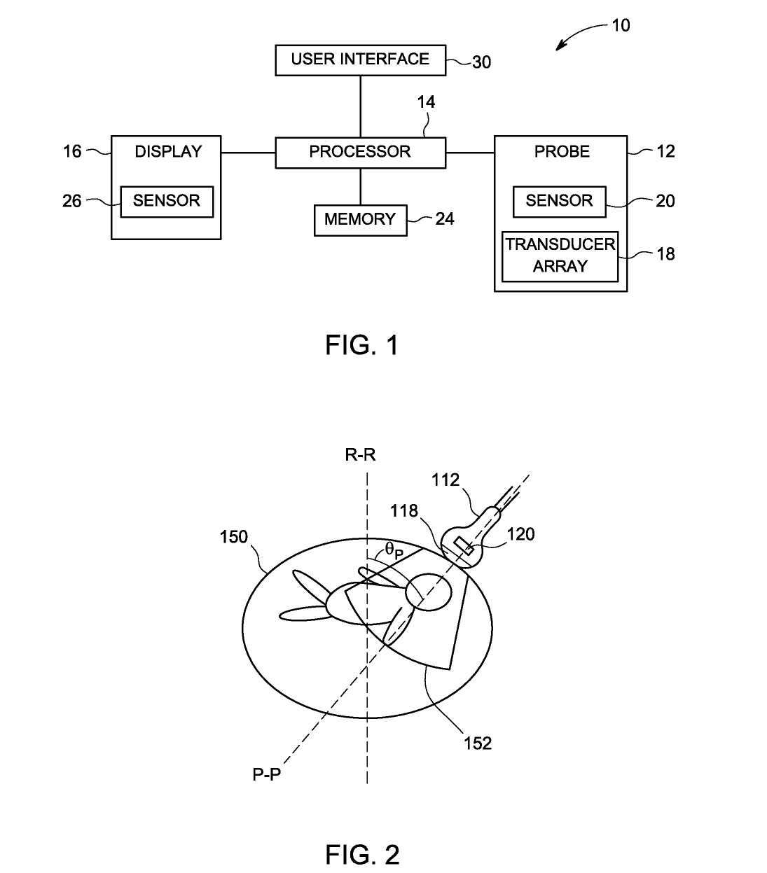

Referring to FIG. 1, an ultrasound system 10 includes a probe 12, a processor 14, and a display 16. Both the probe 12 and the display 16 are operatively connected to processor 14. This connection may be wired or wireless. The ultrasound system 10 may be a console-based or laptop system or a portable system, such as a handheld system. In one embodiment, the processor 14 may be integral to the probe 12. In another embodiment, the processor 14 and the display 16 may be integrated into a single housing.

The probe 12 is configured to transmit ultrasound signals toward a target region of interest (ROI) and receive returned echo data from the target ROI. The probe comprises a transducer array 18. The transducer array 18 has a plurality of transducer elements configured to emit pulsed ultrasonic signals into a target region of interest (ROI). It should be appreciated that while the transducer array may have a variety of geometries including 2D array, curved linear array, and convex array, the transducer array 18 will comprise at least one row of transducer elements.

Pulsed ultrasonic signals are back-scattered from structures in the body, like blood cells or muscular tissue, to produce echoes that return to the transducer array 18. The echoes are converted into electrical signals, or ultrasound data, by the transducer elements in the transducer array 18 and the electrical signals are received by the processor 14.

The probe 12 also may include a probe orientation sensor 20. Orientation sensor 20 is configured to measure a tilt angle of probe 12 with respect to a vertical gravitational axis, for example, axis R-R shown in FIG. 2. The orientation sensor 20 may comprise an accelerometer. An accelerometer is a device that measures static or dynamic acceleration forces. By measuring, for example, the amount of static acceleration due to gravity, an orientation or tilt of a device with respect to the earth can be determined. The orientation sensor 20 may further comprise any other device or technology known to determine the orientation of an object with respect to a vertical gravitational axis. For example, the orientation sensor 20 may comprise optical tracking, electromagnetic field (EMF) tracking or image tracking devices, or any combination thereof.

The processor 14 may be able to control the acquisition of ultrasound data by the probe 12, process the ultrasound data, and generate frames or images for display on the display 16. The processor 14 may, for example, be a central processing unit, a microprocessor, a digital signal processor, or any other electrical component adapted for following logical instructions. The processor 14 may also comprise a tracking technology, as an alternative to, or in addition to orientation sensor 20 in the probe, such as image tracking technology, in order to determine a tilt angle or orientation of probe 12 with respect to a vertical gravitational axis, based on the generated image and movement of the image over time.

The processor 14 may be operatively connected to a memory 24. The memory 24 is a non-transitory computer readable storage medium. The memory 24 is configured to store instructions, programs and ultrasound data such as processed frames of acquired ultrasound data that are not scheduled to be displayed immediately.

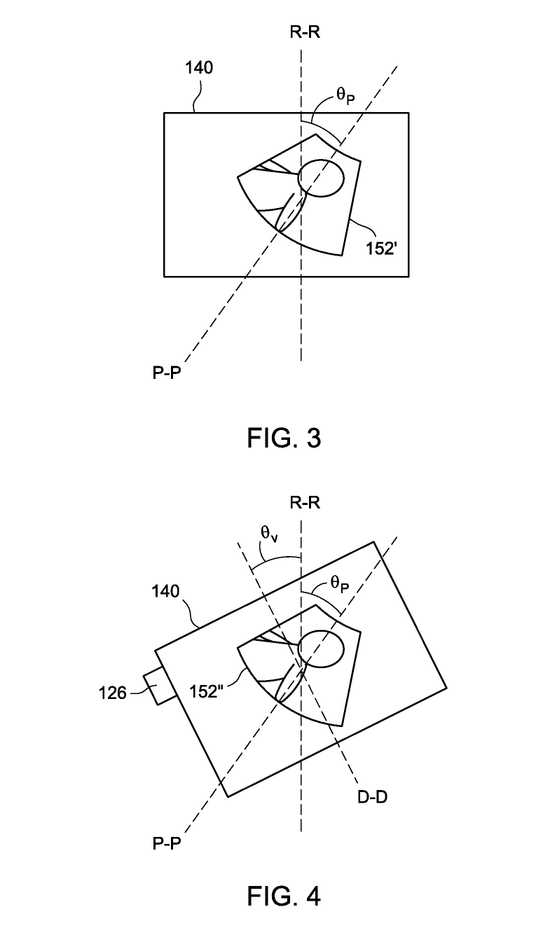

The processor 14 may also be operatively connected to a user interface 30. The user interface 30 may be a series of hard buttons, a plurality of keys forming a keyboard, a trim knob, a touchscreen, or some combination thereof. It should be appreciated that additional embodiments of the user interface 30 may be envisioned. The user interface 30 may be used to control operation of the ultrasound system 10, including to control the input of patient data, to change a scanning or display parameter, and the like. For example, the user interface 30 may configured to allow the ultrasound operator to select between display modes. The display modes may be a standard mode, as described with respect to the prior art, and an orientation-adjusted mode as described herein with respect to FIGS. 3-6.

Display 16 is operatively connected to processor 14 and is configured to display images. Images may be displayed on display 16 in real time. For purposes of this disclosure, the term “real-time” is defined to include a process performed with no intentional lag or delay. An embodiment may update the displayed ultrasound image at a rate of more than 20 times per second. The images may be displayed as part of a live image. For purposes of this disclosure, the term “live image” is defined to include a dynamic image that updates as additional frames of ultrasound data are acquired. For example, ultrasound data may be acquired even as images are being generated based on previously acquired data while a live image is being displayed. As additional ultrasound data are acquired, additional frames or images generated from more recently acquired ultrasound data are sequentially displayed. Additionally and alternatively, images may be displayed on display 16 in less than real time. Ultrasound data may be stored in memory 24 during a scanning session and then processed and displayed at a later time.

The display 16 may have a display orientation sensor 26. Similar to orientation sensor 20, the orientation sensor 26 is configured to measure the tilt angle of display 16 with respect to a vertical gravitational axis, for example, shown in FIG. 4. The orientation sensor 26 may be an accelerometer. It should be appreciated that the orientation sensor 26 may be any other device or technology known to determine the orientation of an object with respect to a vertical gravitational axis. For example, the orientation sensor 26 may be an EMF tracking device.

Referring to FIG. 2, a schematic representation of a target 150 is shown in accordance with an embodiment. Target 150 may be a human or an animal. In the embodiment shown, the target 150 is a pregnant subject. The target 150 is oriented with respect to a reference axis R-R. Reference axis R-R is a vertical gravitational axis. Target 150 comprises a ROI 152. The ROI 152 may be a subset of the target 150. For example, in the embodiment shown, the ROI 152 comprises a fetus within the target 150.

A probe 112 comprises a transducer array 118 and a probe orientation sensor 120. The transducer array 118 is configured to send ultrasonic signal towards a target ROI 152 and receive the resulting echo data. The ROI 152 comprises a center line P-P that bisects the ROI 152. The center line P-P is perpendicular to transducer array 118 and bisects the transducer array 118. For example, if the transducer array 118 comprises a row of 100 transducer elements, the center line P-P bisects the row of transducer elements with 50 transducer elements on either side of center line P-P.

Orientation sensor 120 is configured to determine an angle θ_{P} of probe 112 with respect to axis R-R. Angle θ_{P} is defined by the angle between axis R-R and center line P-P. If probe 112 were aligned with axis R-R, the angle θ_{P} would be 0 degrees and the center line P-P would be aligned with axis R-R. In the depicted embodiment shown, angle θ_{P} is greater than 0 degrees but less than 90 degrees. It should be appreciated that angle θ_{P} may vary from 0 degrees to 180 degrees in either clockwise or counterclockwise direction from axis R-R. Angle θ_{P} can change in real time as the orientation of probe 112 changes.

FIG. 3 comprises a schematic representation of the display 140 in accordance with an embodiment. The display 140 comprises an image of the ROI 152′. The center line P-P of image of ROI 152′ is displayed at the angle θ_{P} with respect to axis R-R. Angle θ_{P} is the same in both FIGS. 2 and 3.

In FIG. 4, the display 140 is shown in accordance with another embodiment. The display 140 has a display orientation sensor 126 that is configured to determine a display orientation, angle θ_{V}, with respect to axis R-R. If the display 140 is level, angle θ_{V} is equal to 0 degrees and a display axis D-D is parallel axis R-R. When the display 140 is tilted with respect to axis R-R, angle θ_{V} is greater than 0 degrees and axis D-D is no longer parallel to axis R-R. For example, in the depicted embodiment, the angle θ_{V} is greater than 0 degrees but less than 90 degrees and axis R-R and axis D-D are not parallel. It should be appreciated that angle θ_{V} may vary from 0 degrees to 180 degrees in either clockwise or counterclockwise direction from axis R-R. Angle θ_{V} can change in real time as the orientation of display 140 changes.

The display 140 has an image of the ROI 152″ displayed with center line P-P at angle θ_{P} with respect to axis R-R and center line P-P at a display angle comprised of the sum of angle θ_{P} and angle θ_{V} with respect to axis D-D. Image of ROI 152″ is also displayed so that angle θ_{P} is the same in both FIGS. 2 and 4, despite angle θ_{V} being greater than 0 degrees. The result is that the image of ROI 152″ does not change from the user's perspective despite the angle θ_{V} of the display 140.

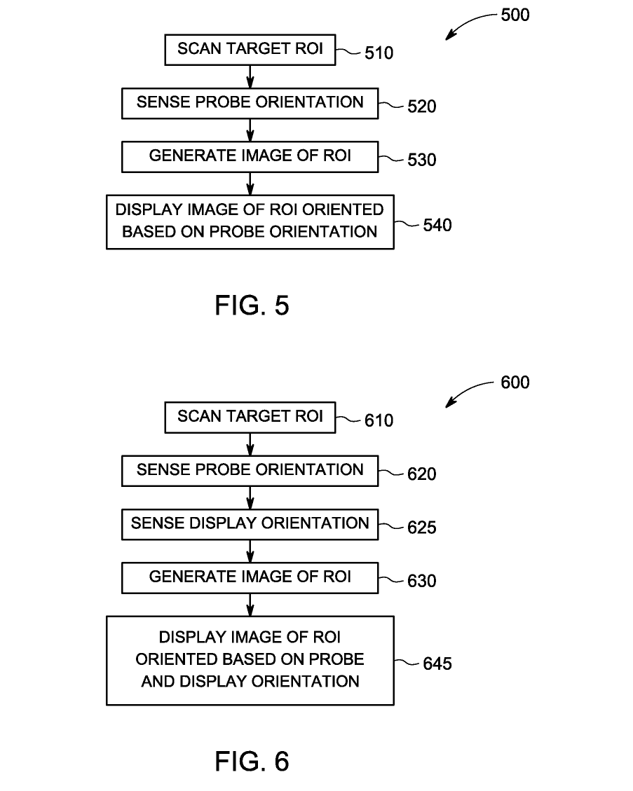

Having described various embodiments of the ultrasound system 10, a method 500 of displaying the ultrasound image will be described in accordance with FIG. 5. Reference numerals will refer to any of the FIGS. 1-6. The method 500 may comprise a step 510 comprising scanning with the probe 112 a target ROI 152 and receiving echo data from the ROI 152. The probe comprises transducer array 118 that is configured to emit pulsed ultrasound signals and receive the backscattered ultrasound signals as echo data. Step 510 may be done according to known techniques in the art.

The method 500 may include a step 520 comprising sensing, with the sensor 120, the probe orientation, angle θ_{P}. The orientation sensor 120 may be an accelerometer, optical tracking, electromagnetic field (EMF) tracking or image tracking device. Sensor 20 may be any other device or technology known to determine the orientation of an object with respect to a vertical gravitational axis. The probe orientation, angle θ_{P}, can change in real time as the probe 112 moves. When the probe 112 is held parallel to vertical gravitational axis R-R, the angle θ_{P} is 0 degrees. However, when the probe 112 moves away from such a parallel position, the angle θ_{P} will be greater than zero.

The method 500 may include a step 530 comprising generating with the processor 14 an image of the ROI 152′. Processor 14 receives ROI echo data from the probe 12 and generates an image of the ROI 152′ according to known techniques in the art.

The method 500 may include a step 540 comprising displaying with the display 16, 140 the image of the ROI 152′ base on the probe orientation, angle θ_{P} with respect to axis R-R. Specifically, center line P-P will be displayed at angle θ_{P} with respect to axis R-R.

The method 500 may also include an additional step comprising selecting with a user interface a display mode. The display mode may be a standard mode or an orientation-adjusted mode. The standard mode is, as described with respect to the prior art, wherein the center line P-P of image 152′ is parallel with reference axis R-R and angle θ_{P} therefore equals 0 degrees. The orientation-adjusted mode is depicted in FIG. 3, wherein the center line P-P of image 152′ is not parallel with reference axis R-R and angle θ_{P} is therefore greater than 0 degrees.

Referring to FIG. 6, a method 600 of displaying the ultrasound image is depicted. Method 600 comprises steps 610, 620 which are respectively similar to the steps 510 and 520 of method 500. Method 600 further includes a step 625 comprising sensing with the orientation sensor 26 a display orientation, angle θ_{V}. The orientation sensor 26 may be an accelerometer or EMF tracking device. Angle θ_{V} is the angle of display axis D-D with respect to reference axis R-R. This step is particularly important when the display 16 of the ultrasound system 10 is portable or handheld and may not be held with a steady orientation throughout an exam. In this case the display axis D-D is often not parallel with reference axis R-R.

Method 600 may include a step 630 comprising generating with the processor 14 an image of the ROI. Step 630 is similar to step 530 of method 500, and may be accomplished according to known techniques in the art.

Method 600 may include a step 645 comprising displaying with the display 140 the image of the ROI 152″ based on the probe orientation angle θ_{P} and the display orientation angle θ_{V}. The display 140 has an image of the ROI 152″ displayed with center line P-P at angle θ_{P} with respect to axis R-R and center line P-P at a display angle comprised of the sum of angle θ_{P} and angle θ_{V} with respect to axis D-D. The result is that the image of ROI 152″ does not change perspective with respect to the user despite the angle θ_{V} of the display 140.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. An ultrasound imaging system, comprising:
a probe configured to transmit ultrasound signals toward a target region of interest (ROI) and receive returned echo data from the target ROI;
a sensor for generating a signal relating to a probe orientation;
a display for displaying an image of the target ROI;
a processor connected to the probe for receiving the echo data and generating the image of the target ROI, the processor further configured to receive the probe orientation signal and display the image of the target ROI based on the probe orientation signal.

2. The ultrasound system of claim 1, wherein the target ROI has a center line having a first angle with respect to a vertical gravitational axis, and the target ROI is displayed on the display with an image center line at the first angle.

3. The system of claim 2, wherein the probe comprises at least one row of transducer elements and the center line of the target ROI bisects the at least one row.

4. The system of claim 1, wherein the probe orientation sensor comprises at least one of an accelerometer, optical tracking, EMF tracking and image tracking devices.

5. The system of claim 2, wherein the image is acquired and displayed in real-time and the image center line changes as the probe orientation signal changes.

6. The system of claim 2, wherein the display comprises a sensor for determining a display orientation.

7. The system of claim 6, wherein the display orientation sensor comprise at least one of an accelerometer or EMF tracking devices.

8. The system of claim 6, wherein the image is acquired and displayed in real-time and a display angle changes as the display orientation changes.

9. The system of claim 1, further comprising a user interface for selecting a display mode.

10. A method of displaying an ultrasound image, comprising:
scanning with a probe a target region of interest (ROI) and receiving echo data from the ROI,
sensing with a sensor a probe orientation,
generating with a processor an image of the ROI, and
displaying with a display the image of the ROI based on the probe orientation.

11. The method of claim 10, wherein the target ROI has a center line having a first angle with respect to a vertical gravitational axis, and the target ROI is displayed on the display with an image center line at the first angle.

12. The method of claim 11, wherein the probe comprises at least one row of transducers and the center line of the target ROI bisects the at least one row.

13. The method of claim 10, wherein the probe orientation sensor comprises at least one of an accelerometer, optical tracking, EMF tracking and image tracking devices.

14. The method of claim 11, wherein the image center line changes as the probe orientation changes.

15. The method of claim 14, wherein the image is generated and displayed in real-time.

16. The method of claim 10, further comprising:
sensing with a second sensor a display orientation.

17. The method of claim 16, wherein the second sensor comprises at least one of an accelerometer and EMF tracking devices.

18. The method of claim 17, wherein the displaying step is based on the probe orientation and the display orientation.

19. The method of claim 17, wherein the image is generated and displayed in real-time.

20. The method of claim 10, further comprising:
selecting with a user interface a display mode.
